(19) United States
(12) Patent Application Publication
(10) Pub. No.: US 2018/0204028 A1
(43) Pub. Date: Jul. 19, 2018

(54) APPARATUS AND METHOD FOR RECORDING ULTRASONIC IMAGE

(71) Applicant: Jin Pyo CHOI, Seoul (KR)

(72) Inventor: Jin Pyo CHOI, Seoul (KR)

(21) Appl. No.: 15/744,696

(22) PCT Filed: Jul. 13, 2016
(86) PCT No.: PCT/KR2016/007629

§ 371 Date: Jan. 12, 2018

(30) Foreign Application Priority Data
Jul. 13, 2015 (KR) 10-2015-0099060

(51) Int. Cl. G06K 7/10; A61B 8/00; A61B 8/08

(52) U.S. Cl. CPC G06K 7/10; A61B 8/08; A61B 8/565

(57) ABSTRACT

Disclosed are an apparatus and a method for recording an ultrasonic image, which are capable of automatically recording an ultrasonic image acquired by an ultrasonic image diagnosis apparatus. An apparatus for recording an ultrasonic image according to one embodiment comprises: a recognition unit configured to recognize identification information of a patient; a first communication unit configured to receive an ultrasonic image from an ultrasonic image diagnosis apparatus, the first communication unit being electrically coupled to the ultrasonic image diagnosis apparatus; a control unit configured to automatically record the ultrasonic image in the case where a record initiation command for recording the ultrasonic image is not inputted within a predetermined time; and a second communication unit configured to transmit, to a server, the recorded ultrasonic image and the identification information mapped to the ultrasonic image.

### TECHNICAL FIELD

The present invention relates to an apparatus and a method for recording ultrasonic images, more particularly, to an apparatus and a method for recording an ultrasonic image, which can automatically record the ultrasonic image in connection with an ultrasonic image diagnostic device.

### BACKGROUND ART

Medical image equipment may include, for example, an X-ray imaging system, an X-ray fluoroscopy system, a computed tomography scanner (CT), a magnetic resonance image system (MRI), a positron emission tomography (PET) system, an ultrasonic image diagnostic device, or the like.

The ultrasonic image diagnostic device is an apparatus that irradiates a subject with ultrasonic waves and non-invasively obtains tomograms of internal tissues or images of blood flow on the basis of ultrasonic echoes reflected from the inside of the subject.

The ultrasonic image diagnostic device has advantages such as a smaller size, lower price and possibility of real-time display, or the like, as compared to other medical imaging apparatuses. Further, there is no risk of exposing a patient to radiation such as X-rays to thus improve stability. Accordingly, the ultrasonic image diagnostic device is now utilized in a wide range of applications including diagnosis of the heart, the breasts, the abdomen, and the urinary tract, and for obstetric and gynecologic purposes.

In general, ultrasonic diagnosis is performed by an ultrasonic diagnostician (‘sonographer’). More particularly, the ultrasonic diagnosis may be conducted by the sonographer who manipulates a probe with one hand while operating an input unit with the other hand.

In order to record ultrasonic images during ultrasonic diagnosis, the sonographer should input a command to record the images (‘recording command’). However, it is quite cumbersome and inconvenient to input such a recording command while manipulating the probe.

### DISCLOSURE

### Technical Problem

Disclosed are an apparatus and a method for recording ultrasonic images which can automatically record ultrasonic images in connection with an ultrasonic image diagnostic device.

### Technical Problem

In order to accomplish the above object, in accordance with an embodiment of the present invention, provided is an ultrasonic image recording apparatus which includes: a recognition unit configured to recognize identification information of a patient; a first communication unit configured to receive an ultrasonic image from an ultrasonic image diagnostic device, which is electrically coupled to the ultrasonic image diagnostic device; a control unit configured to automatically record the ultrasonic image in case where a recording execution command for recording the ultrasonic image is not input within a (predetermined) reference time; and a second communication unit configured to transmit the recorded ultrasonic image and the identification information mapped to the ultrasonic image, to a server.

The control unit executes recording of the ultrasonic image if a quality of the ultrasonic image is greater than or equal to a reference quality, and outputs a guide message indicating that the ultrasonic image is automatically recorded.

The control unit determines that the quality of the ultrasonic image is greater than or equal to the reference quality when speckle noise is not contained in the ultrasonic image or a boundary between tissues is clearly displayed in the ultrasonic image.

The control unit executes the recording of the ultrasonic image when motion is detected in the ultrasonic image, and then outputs a guide message informing that the ultrasonic image is automatically recorded.

The recognition unit includes at least one bar code reader which recognizes the identification information from a bar code possessed by a patient, a wireless signal transceiver which transmits a wireless signal to and receives the same from a first device possessed by the patient, so as to gain the identification information.

### Advantageous Effects

Since the ultrasonic image can be recorded without any separate operation during the ultrasonic diagnosis, improved user convenience in using the ultrasonic image diagnostic device may be achieved.

### DESCRIPTION OF DRAWINGS

FIG. 1 illustrates an ultrasonic image recording system according to one embodiment of the present invention;

FIG. 2 is a view illustrating the appearance of an ultrasonic image diagnostic device shown in FIG. 1.

FIG. 3 is a view illustrating the configuration of the ultrasonic image diagnostic device shown in FIG. 1.

FIG. 4 is a view illustrating the configuration of an ultrasonic image recording apparatus shown in FIG. 1.

FIG. 5 is a flowchart illustrating an ultrasonic image recording method according to one embodiment of the present invention.

FIG. 6 is a flowchart illustrating step S540 of FIG. 5 in more detail.

### BEST MODE

The advantages and features of the present invention, and a method of accomplishing the same will be more apparent from the following detailed description when taken in conjunction with the accompanying drawings. The present invention may, however, be embodied in a variety of different forms and should not be construed as being limited to the embodiments set forth herein. Instead, the present examples are provided only to complete the description of the present invention and to clearly explain the scope of the present invention to a person having ordinary skill in the art (′one skilled in the art′), to which the present invention pertains. Therefore, the scope of present invention is defined only by the appended claims.

Unless otherwise defined, all terms (including technical and scientific terms) used in the present invention can be used in a sense that is commonly understood by one skilled in the art. Further, terms defined in commonly-used dictionaries may not be ideally or excessively construed unless obviously and specifically defined.

The terms used herein are for the purpose of describing particular embodiments only and are not intended to limit the invention. In the present specification, the singular forms “a”, “an” and “the” include plural referents unless context clearly indicates otherwise. As used herein, the terms “comprises” and/or “comprising” in the present invention do not exclude the presence or addition of one or more other components in addition to the components described above.

Hereinafter, in conjunction with the accompanying drawings, preferred embodiments of the present invention will be described in detail. The same symbols in the drawings denote the same components.

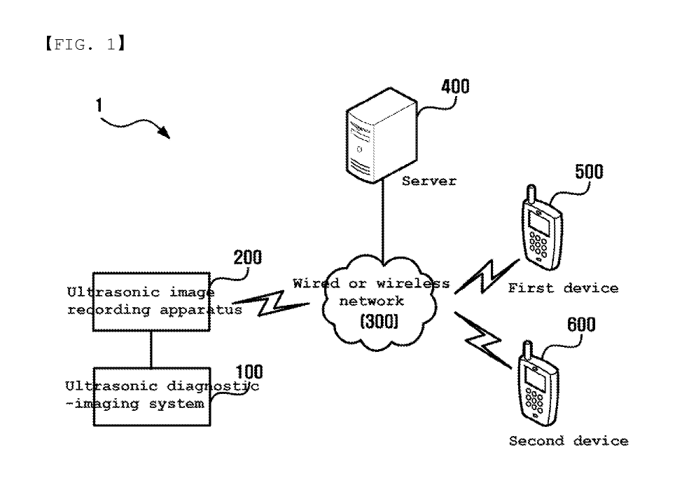

FIG. 1 is a view illustrating the configuration of an ultrasonic image recording system 1 according to an embodiment of the present invention.

Referring to FIG. 1, the ultrasonic image recording system 1 according to the embodiment of the present invention includes an ultrasonic image diagnostic device 100, an ultrasonic image recording apparatus 200, a server 400, a first device 500 and a second device 600.

The ultrasonic image diagnostic device 100 is an apparatus that radiates ultrasonic waves toward a target site in a subject and non-invasively obtains tomograms of soft tissues or images of blood flow by using information of the ultrasonic waves (ultrasonic echoes) reflected from the target site.

The ultrasonic image acquired by the ultrasonic image diagnostic device 100 may be a two-dimensional ultrasonic image or a three-dimensional ultrasonic image. The two-dimensional ultrasonic image refers to a cross-sectional image of internal tissue in the subject. The three-dimensional ultrasonic image refers to an image obtained by volume-rendering three-dimensional volume data which are formed on the basis of a plurality of cross-sectional images.

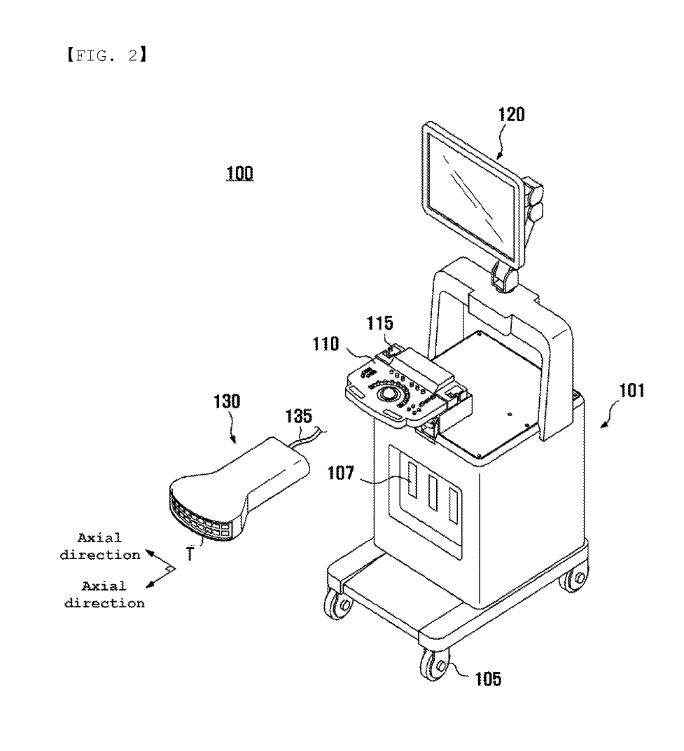

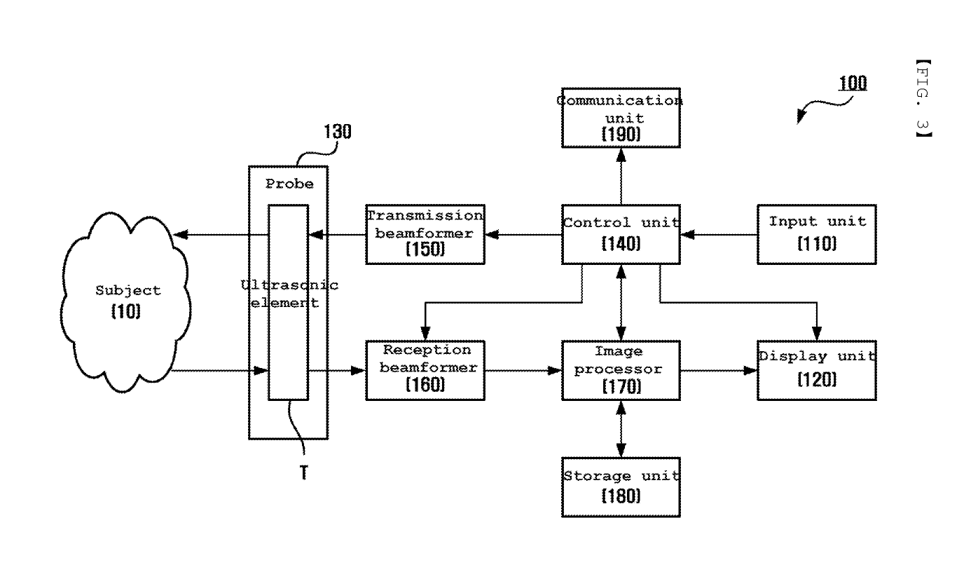

The ultrasonic image diagnostic device 100 may display the two-dimensional ultrasonic image or the three-dimensional ultrasonic image as a black and white image or a color image. The color image may be obtained by mapping colors similar to the internal tissues of the subject to the black and white image. Hereinafter, appearance and configuration of the ultrasonic image diagnostic device 100 will be more concretely stipulated with reference to FIGS. 2 and 3.

The ultrasonic image recording apparatus 200 is electrically coupled to the ultrasonic image diagnostic device 100. More particularly, the ultrasonic image recording apparatus 200 may have a communication port (not shown) and this communication port may be mechanically coupled with another communication port (not shown) provided in the ultrasonic image diagnostic device 100.

According to an embodiment of the present invention, the ultrasonic image recording apparatus 200 may recognize identification information of a patient. The identification information of a patient may include at least one of name, gender, age, address and/or contact information (mobile phone, e-mail), etc. Such identification information may be, for example, stored in a bar code or the first device 500 of the patient.

If the identification information of the patient is recognized, the ultrasonic image recording apparatus 200 records an ultrasonic image received from the ultrasonic image diagnostic device 100. More particularly, if a recording execution command is input within a reference time, the ultrasonic image recording apparatus 200 executes recording of the ultrasonic image. According to the embodiment, even if the recording execution command is not input within the reference time, the ultrasonic image recording apparatus 200 may record the ultrasonic image received from the ultrasonic image diagnostic device 100. The identification information of the patient may be mapped to the recorded ultrasonic image.

Further, the ultrasonic image recording apparatus 200 periodically checks the state of ultrasonic image being recorded. As a result of the checking, if the ultrasonic image is abnormal, the ultrasonic image recording apparatus 200 transmits a guide message to announce occurrence of abnormality in the ultrasonic image to a server 400 through a wired or wireless network.

Thereafter, the ultrasonic image recording apparatus 200 determines whether ultrasonic diagnosis has ended. For example, if no further ultrasonic image is received from the ultrasonic image diagnostic device 100, the ultrasonic image recording apparatus 200 determines that ultrasonic diagnosis has ended. In another example, if the ultrasonic image received from the ultrasonic image diagnostic device 100 is not substantially altered, as compared to the previously received ultrasonic image, the ultrasonic image recording apparatus 200 determines that ultrasonic diagnosis has ended. In another example, if the ultrasonic image diagnostic device 100 is powered off, the ultrasonic image recording apparatus 200 determines that ultrasonic diagnosis has ended. In a further example, if a recording shutdown command is input through the ultrasonic image diagnostic device 100 or the ultrasonic imaging recording apparatus 200, the ultrasonic image recording apparatus 200 determines that ultrasonic diagnosis has ended. Upon determining that ultrasonic diagnosis has ended, the ultrasonic image recording apparatus 200 stops recording and then transmits the recorded ultrasonic images to a server 400 through a wired and/or wireless network.

The server 400 communicates with the ultrasonic image recording apparatus 200 through a wired and/or wireless network. For example, the server 400 receives a result of checking whether or not the recorded ultrasonic image is abnormal from the ultrasonic image recording apparatus 200. In another example, the server 400 receives the completely recorded ultrasonic image from the ultrasonic image recording apparatus 200.

Identification information of a patient is mapped to the completely recorded ultrasonic image. More particularly, the server 400 may sort and store ultrasonic images received from the ultrasonic image recording apparatus 200 on the basis of the identification information of the patient. The ultrasonic image stored in the server 400 may be subjected to streaming or downloading onto the first device 500 or the second device 600.

The server 400 may store and handle information about the first device 500 and/or the second device 600. The information about the first device 500 and/or the second device 600 may be registered in the server 400 beforehand.

The first device 500 refers to a digital device used by the patient. The second device 600 refers to a digital device used by any person with a social relationship with the patient (e.g., family of the patient, any friend of the patient). The first device 500 and the second device 600 may include a wired and/or wireless communication device. The wired and/or wireless communication device may include, for example, a smartphone, tablet and personal computer (PC), etc. However, the wired and/or wireless communication device is not particularly limited to those exemplified herein.

FIG. 2 is a view illustrating the appearance of the ultrasonic image diagnostic device 100 shown in FIG. 1, while FIG. 3 is a view illustrating the configuration of the ultrasonic image diagnostic device 100 shown in FIG. 1.

Referring to FIG. 2, the ultrasonic image diagnostic device 100 may include a main body 101, an input unit 110, a display unit 120 and a probe 130.

Major components of the ultrasonic image diagnostic device 100 are loaded in the main body 101. For instance, as shown in FIG. 3, a control unit 140, a transmission beamformer 150, a reception beamformer 160, an image processor 170, a storage unit 180 and a communication unit 190, etc. may be loaded in the main body 101. These components will be more concretely described below with reference to FIG. 3.

A female connector 107 is provided at one side of the main body 101. The female connector 107 may be mechanically coupled with a male connector (not shown). The male connector may be connected to one end of a cable 135, while providing the probe 130 at the other end of the cable 135.

On bottom of the main body 101, multiple casters 105 are provided for mobility of the ultrasonic image diagnostic device 100. These multiple casters 105 may serve to fix the ultrasonic image diagnostic device 100 at a specific site or move the same in a specific direction.

The input unit 110 is a section wherein a sonographer can input a command or information required for ultrasonic diagnosis. For example, the sonographer may operate the input unit 110 to input a mode selection command in relation to images, a recording execution command, or the like. In another example, the sonographer may operate the input unit 110 to set different setting values required for actuating the ultrasonic image recording apparatus 200. For example, output of a guide message, a reference time for automatically recording ultrasonic images, a recording reference for ultrasonic images, etc. may be set beforehand. For this purpose, the input unit 110 may include, for example, at least one among a keyboard, a mouse, a foot switch and a foot pedal.

The keyboard may be configured in a hardware mode and may include at least one among a switch, a key, a wheel, a joystick, a trackball and a knob. The keyboard may be arranged on an upper part of the main body. In another example, the keyboard may be implemented in a software mode such as a graphical user interface. Such a software keyboard may be displayed through a display unit. A foot switch or a foot pedal may be disposed at a lower part of the main body, and the sonographer may control some functions of the ultrasonic image diagnostic device 100 by using the foot switch or foot pedal.

One or more probe holders 115 for holding the probe 130 may be provided at one side of the input unit 110. A shape and size of each probe holder 115 may be the same or different.

The display unit 120 displays the ultrasonic image. The display unit 120 may have only a single display function, or otherwise, may have both of a display function and an input function. For example, when the display unit is implemented as a touch screen, the display unit 120 may be considered to have both of the display function and the input function.

The probe 130 is a part which is in contact with the skin of a subject (see 10 in FIG. 3). At an end of the probe 130, at least one ultrasonic element T and a cover covering the at least one ultrasonic element T are provided.

The at least one ultrasonic element T radiates ultrasonic waves to a target site in the subject 10, receives ultrasonic echoes reflected from the target site and converts the same into an electrical signal. According to an embodiment of the present invention, the ultrasonic element T may include an ultrasonic wave generating element to generate ultrasonic waves and an ultrasonic wave receiving element to receive ultrasonic echoes and convert the same into an electrical signal. According to another embodiment of the present invention, ultrasonic wave generation and ultrasonic echo reception may be performed in a single ultrasonic element T.

The ultrasonic element T may be an ultrasonic transducer. A transducer refers to a device for converting a specific form of energy into another type of energy. For example, an ultrasonic transducer may convert electrical energy into wave energy and convert wave energy into electrical energy. In other words, the ultrasonic transducer may perform functions of the ultrasonic wave generating element and functions of the ultrasonic receiving element.

More particularly, the ultrasonic transducer may include a piezoelectric material or piezoelectric thin film. If alternating current is applied to a piezoelectric material or a piezoelectric thin film from an internal electric condenser such as a battery or an external power supply, the piezoelectric material or the piezoelectric thin film is vibrated at a predetermined frequency and an ultrasonic wave having a predetermined frequency is generated according to the vibration frequency. In contrast, when the ultrasonic echo at a predetermined frequency reaches the piezoelectric material or the piezoelectric thin film, the piezoelectric material or the piezoelectric thin film vibrates according to the frequency of the ultrasonic echo to which the piezoelectric material or the piezoelectric thin film has reached. At this time, the piezoelectric material or the piezoelectric thin film outputs alternating current at a frequency corresponding to the vibration frequency.

The ultrasonic transducer used herein may include a variety of ultrasonic transducers, for example, a magnetostrictive ultrasonic transducer using magnetostrictive effects of a magnetic material, a piezoelectric ultrasonic transducer using the piezoelectric effect of a piezoelectric material, a capacitive micromachined ultrasonic transducer (cMUT) to transmit and receive ultrasonic waves using vibration of hundreds or thousands of micromachined thin films, or the like. In addition, other types of transducers capable of generating an ultrasonic wave by an electrical signal or generating an electrical signal by an ultrasonic wave may also be used as the ultrasonic transducer.

The at least one ultrasonic transducer may be arranged in a linear form (linear array) or arranged in a curved form (convex array) at the end of the probe 130. At this time, the at least one ultrasonic transducer may be arranged in a linear form or in a matrix form. In a case where at least one ultrasonic transducer is arranged in a linear form, the probe 130 may be moved in a scanning direction to obtain a plurality of ultrasonic images. When the at least one ultrasonic transducer is arranged in a matrix, a plurality of ultrasonic images can be acquired by ultrasonic transmission at one time.

Referring to FIG. 3, the control unit 140 controls general motions of the ultrasonic image diagnostic device 100. More particularly, the control unit 140 may generate a control signal to control at least one among a transmission beamformer 150, a reception beamformer 160, an image processor 170 and the display unit 120, in response to instruction or command input through the input unit 110. According to an embodiment of the present invention, the control unit 140 may generate a control signal to control each of components in response to an instruction or commend received from an external device (not shown) through wired or wireless communication. In this case, the ultrasonic image diagnostic device 100 may further include any communication unit (not shown) to receive the instruction or command from the external device.

The transmission beamformer 150 may execute transmission beamforming. Transmission beamforming refers to focusing ultrasonic waves generated in at least one ultrasonic element T on a focal point.

The reception beamformer 160 may execute reception beamforming on an ultrasonic signal converted into a digital signal. The reception beamforming refers to compensating for a time difference between ultrasonic signals output from each ultrasonic element and then focusing the same. The focused ultrasonic signal can be understood to be a cross-sectional image with respect to the subject 10. Such a cross-sectional image is provided to the image processor 170.

The storage unit 180 stores data or algorithms required to actuate the ultrasonic image diagnostic device 100. Further, the storage unit 180 stores ultrasonic images acquired during ultrasonic diagnosis. Such a storage unit 180 may include a non-volatile memory, a volatile memory, a hard disk drive, an optical disc drive or a combination thereof.

The image processor 170 generates an ultrasonic image based on an ultrasonic signal focused by the reception beamformer 160. When the ultrasonic signal is focused on a single frame in the reception beamformer 160, one ultrasonic image is generated. If an ultrasonic signal for multiple frames is focused on the reception beamformer 160, a plurality of ultrasonic images is generated. Herein, the plurality of ultrasonic images may be understood to be volume data for the subject 10.

The image processor 170 executes volume rendering according to any volume rendering method known in the art. When volume rendering is completed, a black and white projection image or a color projection image is displayed to the sonographer. Types of the image obtained as a result of volume rendering may be selected by the sonographer before executing ultrasonic diagnosis. The selected value may be changed during ultrasonic diagnosis.

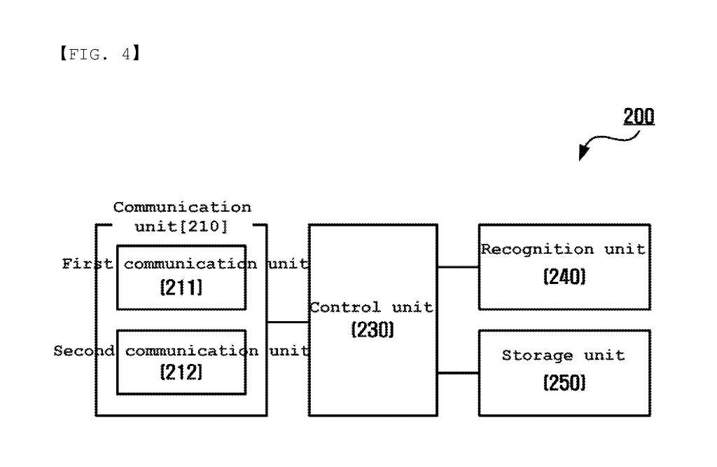

FIG. 4 is a view illustrating the configuration of the ultrasonic image recording apparatus 200 shown in FIG. 1.

Referring to FIG. 4, the ultrasonic image recording apparatus 200 according to one embodiment of the present invention includes a communication unit 210, a control unit 230, a recognition unit 240 and a storage unit 250.

The communication unit 210 includes a first communication unit 211 and a second communication unit 212. The first communication unit 211 communicates with the ultrasonic image diagnostic device 100. More particularly, the first communication unit 211 receives an ultrasonic image from the ultrasonic image diagnostic device 100. The first communication unit 211 may support, for example, a wired communication mode. Although not shown in the drawings, the first communication unit further includes a communication port. The communication port of the first communication unit 211 is electrically coupled to another communication port included in the ultrasonic image diagnostic device 100.

The second communication unit 212 communicates with the server 400. More particularly, the second communication unit 212 transmits the recorded ultrasonic image to the server 400. The second communication unit 212 may support a wired communication mode and/or a wireless communication mode. The wireless communication mode may include, for example, ultra-wide band, Wi-Fi, Bluetooth, ZigBee, radio frequency (RF) and infrared data association (IrDA).

The recognition unit 240 may recognize identification information of a patient and provide the identification information to a control unit 230 described below. According to an embodiment of the present invention, the recognition unit 240 may include a bar code reader. In this case, the recognition unit 240 recognizes identification information of a patient from the bar code possessed by the patient. The bar code may be issued when a patient makes an appointment for or applies for medical treatment. Further, the bar code may be issued in the form of printed media or in an image format capable of being stored in the first device 500. The bar code issued in the form of printed media is a sticker form which can be attached to a part of the body of a user or a patient's abdomen, and can be worn on a part of the body of the user (e.g., wrist, ankle, etc.).

According to another embodiment of the present invention, the recognition unit 240 may include a wireless signal transceiver. The wireless signal transceiver may be, for example, a beacon signal transceiver to transmit and receive a beacon signal. Accordingly, the wireless signal transceiver periodically generates and transmits a wireless signal. Therefore, when a patient carrying the first device 500 enters a wireless signal transmission area of the wireless signal transceiver, the first device 500 receives the wireless signal and then transmits identification information of the patient to the wireless signal transceiver in response to the received wireless signal.

The control unit 230 determines whether a recording execution command is input in a reference time after recognizing the identification information of the patient. At this time, the reference time may be set beforehand. The set reference time may be implemented without being changed, or otherwise, may be embodied to be modified by the sonographer. As shown in FIG. 4, if an ultrasonic image recording apparatus 200 is not equipped with an input means, the sonographer may operate the input unit 110 of the ultrasonic image diagnostic device 100 to set a reference time. If an alternative input means (not shown) is provided in the ultrasonic image recording apparatus 200, the sonographer may set the reference time using the input means.

As a result of the determination, if a recording execution command is input within the reference time, the control unit 230 records the ultrasonic image received from the ultrasonic image diagnostic device 100. As a result of the determination, if a recording execution command is not input within the reference time, the control unit 230 outputs a guide message announcing that the ultrasonic image is automatically recorded, and then, automatically records the ultrasonic image received from the ultrasonic image diagnostic device 100.

Before automatically recording the ultrasonic image, the control unit 230 may evaluate whether a quality of the ultrasonic image is greater than or equal to a reference quality. Criteria for evaluating the quality of an ultrasonic image may include, for example, noise and sharpness.

The ultrasonic image may contain speckle noise in the form of spots due to interference of ultrasonic waves. If the speckle noise is not contained in the ultrasonic image, a quality of the corresponding ultrasonic image may be estimated to be greater than or equal to a reference quality. Noise pattern information corresponding to the speckle noise may be stored in a storage unit 250 of the ultrasonic image recording apparatus beforehand 200. The control unit 230 analyzes the ultrasonic image received from the ultrasonic image diagnostic device 100 and obtains a noise pattern and, if the acquired noise pattern matches the noise pattern information corresponding to pre-stored speckle noise, the corresponding ultrasonic image may be judged to include speckle noise. In addition to the noise pattern information corresponding to the speckle noise, noise pattern information corresponding to a different kind of noise may also be stored in the storage unit 250.

Further, when a boundary between tissues is clearly displayed in the ultrasonic image, the quality of the corresponding ultrasonic image may be estimated to be greater than or equal to the reference quality. In the above description, speckle noise and sharpness are specified as examples of the criteria for quality evaluation of an ultrasonic image, quality evaluation criteria are not necessarily limited to those exemplified above.

When quality evaluation of the ultrasonic image is completed, the control unit 230 may determine whether motion is detected in the ultrasonic image. As a result of the determination, when motion is detected in the ultrasonic image, the control unit 230 may execute recording of the corresponding ultrasonic image. The recorded ultrasonic image may be stored in the storage unit 250.

After completing recording, the control unit 230 maps identification information of a patient to the recorded ultrasonic image. Thereafter, the control unit 230 may transmit the recorded ultrasonic image and the identification information of the patient to the server 400 through a wired or wireless network.

The storage unit 250 stores data or algorithms required for operating the ultrasonic image recording apparatus 200. For example, an algorithm for detecting speckle noise in an ultrasonic image, an algorithm for evaluating the sharpness of the ultrasonic image and an algorithm for detecting motion in the ultrasonic image may be stored. In another example, the storage unit 250 stores the recorded ultrasonic image. The storage unit 250 may include, for example, a non-volatile memory, a volatile memory, a hard disk drive, an optical disc drive, a magneto-optical disc drive, and a combination thereof.

The ultrasonic image recording apparatus 200 may further include an output means for outputting a guide message in addition to the above described components. The output means may include at least one of a sound output unit, an image output unit, an optical output unit and a vibration output unit. If the output unit is not provided in the ultrasonic image recording apparatus 200, the guide message may be output through an output means (e.g., display unit) provided in the ultrasonic image diagnostic device 100.

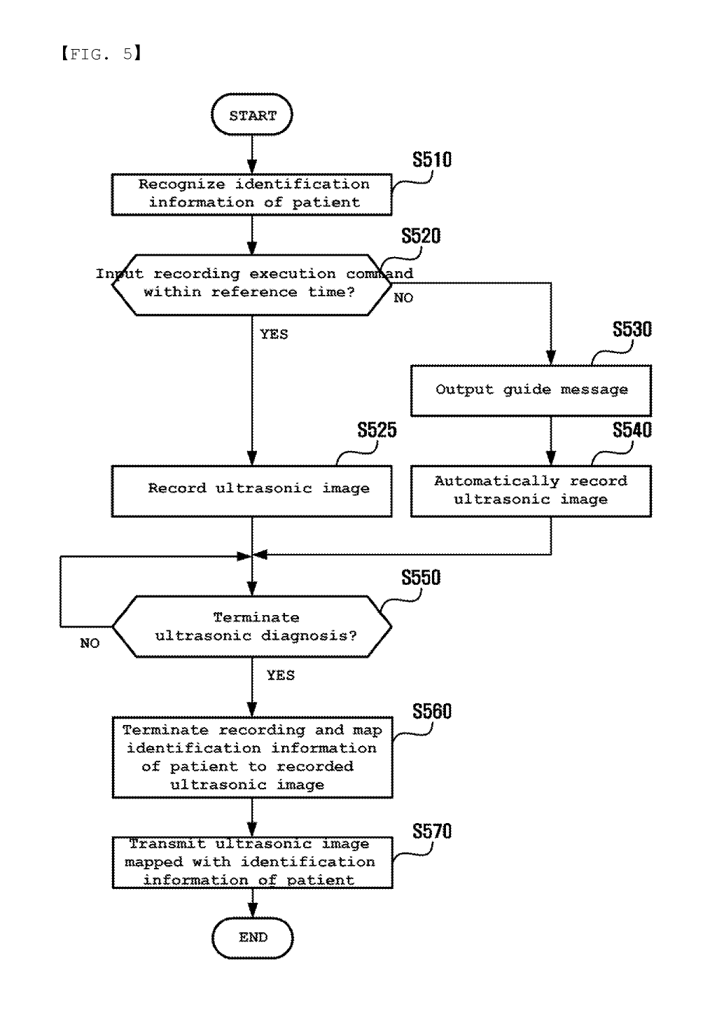

FIG. 5 is a flowchart illustrating a method for recording ultrasonic images according to an embodiment of the present invention.

Prior to the description, it is assumed that the ultrasonic image diagnostic device 100 and the ultrasonic image recording apparatus 200 are included in a medical room, and the ultrasonic image recording apparatus 200 is electrically coupled to the ultrasonic image diagnostic device 100. Further, it is assumed that various set values required to operate the ultrasonic image recording apparatus 200, for example, whether the guide message is output or not, a reference time for automatically recording ultrasonic images, a reference for recording the ultrasonic message, etc., are already set.

First, the ultrasonic image recording apparatus 200 recognizes identification information of a patient (S510). For example, the ultrasonic image recording apparatus 200 recognizes the identification information of a patient from a bar code possessed by the patient. In another example, the ultrasonic image recording apparatus 200 may transmit a wireless signal to the first device 500 possessed by a patient so as to request identification information of the patient, and then, receives the identification information from the first device 500 in response to the transmission described above.

Following this, the ultrasonic image recording apparatus 200 determines whether a recording execution command is input within a reference time (S520).

As a result of the determination in step S520, when the recording execution command is input within the reference time, the ultrasonic image recording apparatus 200 records the ultrasonic image received from the ultrasonic image diagnostic device 100 (S525).

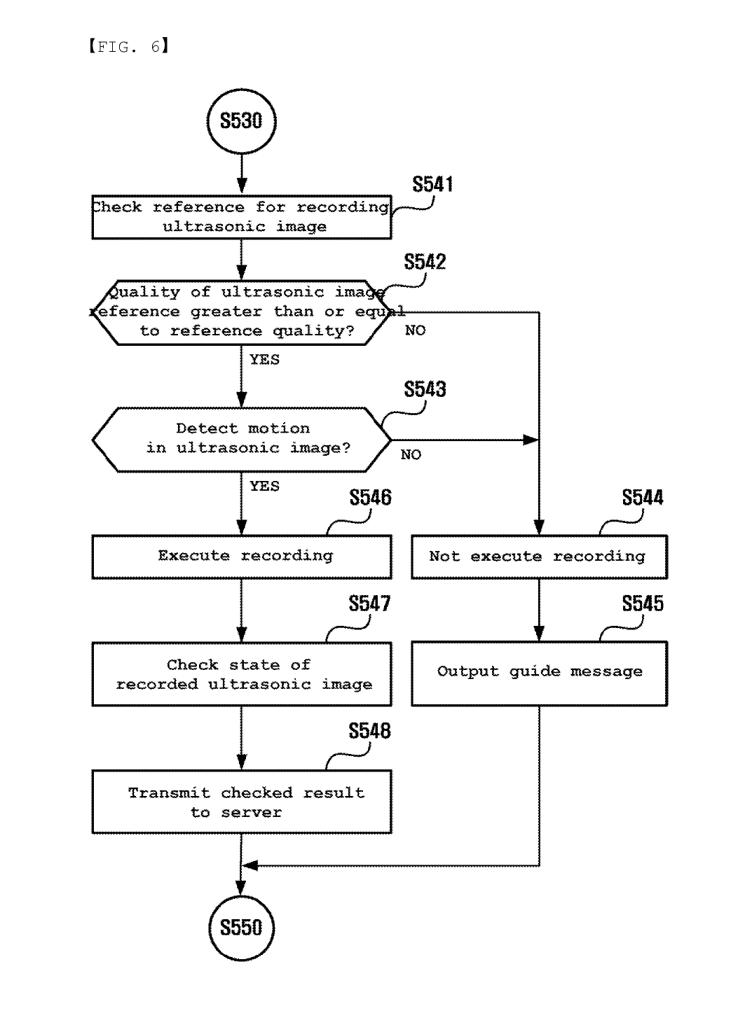

As a result of the determination in step S520, even if the recording execution command is not input within the reference time, the ultrasonic image recording apparatus 200 outputs a guide message announcing that the ultrasonic image is automatically recorded (S530), and then, automatically records the ultrasonic image received from the ultrasonic image diagnostic device 100 (S540). According to an embodiment of the present invention, the ultrasonic image recording apparatus 200 records the corresponding ultrasonic image only when the ultrasonic image satisfies predetermined conditions. A more detailed description of step S520 will be given below with reference to FIG. 6.

Hereinafter, the ultrasonic image recording apparatus 200 determines whether ultrasonic diagnosis has ended (S550). In a case where a recording termination command is input through the ultrasonic image diagnostic device 100 or the ultrasonic image recording apparatus 200, the ultrasonic image recording apparatus 200 may determine that ultrasonic diagnosis has ended. In another case where the ultrasonic image is no longer received from the ultrasonic image diagnostic device 100, the ultrasonic image recording apparatus 200 may determine that ultrasonic diagnosis has ended. In a further case where the ultrasonic image received from the ultrasonic image diagnostic device 100 is substantially not changed as compared to the previously received ultrasonic image, the ultrasonic image recording apparatus 200 may determine that ultrasonic diagnosis has ended.

As a result of the determination in step S550, if it is determined that ultrasonic diagnosis has ended, the ultrasonic image recoding apparatus 200 terminates recording of ultrasonic images and executes mapping of the identification information of the patient (S560). Next, the ultrasonic image recording apparatus 200 transmits the ultrasonic image, to which the identification information of the patient is mapped, to the server 400 through a wired or wireless network (S570).

FIG. 6 is a flowchart illustrating step S540 of automatically recording the ultrasonic image shown in FIG. 5.

First, the ultrasonic image recording apparatus 200 checks a recording reference for the ultrasonic image (S541). The recording reference may include, for example, a quality of the ultrasonic image and whether motion is detected in the ultrasonic image, but is not limited to those exemplified above. The recording reference may be preset by a sonographer. The set recording reference may be embodied without being changed, or otherwise, may be modified by the sonographer.

After checking the recording reference for the ultrasonic image, the ultrasonic image recording apparatus 200 determines that a quality of the ultrasonic image received from the ultrasonic image diagnostic device 100 is greater than or equal to a reference quality (S542). Criteria for evaluating the quality of the ultrasonic image may include, for example, speckle noise and sharpness. Specifically, if the ultrasonic image does not include speckle noise, the quality of the corresponding ultrasonic image may be determined to be greater than or equal to the reference quality. In addition, when a boundary between tissues is clearly displayed in the ultrasonic image, the quality of the corresponding ultrasonic image may be determined to be greater than or equal to the reference quality.

As a result of the determination in step S542, if the quality of the ultrasonic image does not reach the reference quality, the ultrasonic image recording apparatus 200 does not execute recording (S544). Then, a guide message announcing that recording is not executed, is output (S545).

As a result of the determination in step S542, if the quality of the ultrasonic image is greater than or equal to the reference quality, the ultrasonic image recording apparatus 200 determines whether motion is detected in the ultrasonic image (S543). This step S543 may include a step of comparing the ultrasonic image in the previous frame with the ultrasonic image in the current frame, and a step of determining that there is motion if a difference between both the ultrasonic images is equal to or greater than a reference value.

As a result of the determination in step S543, if motion is not detected in the ultrasonic image, the ultrasonic image recording apparatus 200 does not execute recording (S544) but outputs a guide message announcing that recording is not executed (S545). Herein, the guide message may be output in the form of a visual signal, audible signal, tactile signal, or a combination thereof. When an output unit is provided in the ultrasonic image recording apparatus 200, the guide message is output through the output unit of the ultrasonic image recording apparatus 200. If the output unit is not provided in the ultrasonic image recording apparatus 200, the guide message is output through an output unit of the ultrasonic image diagnostic device 100, for example, the display unit 120.

As a result of the determination in step S543, if motion is detected in the ultrasonic image, the ultrasonic image recording apparatus 200 executes recording of the ultrasonic image (S546).

Thereafter, the ultrasonic image recording apparatus 200 checks the state of the ultrasonic image (S547) during recording, and transmits the checked result to the server 400 through a wired or wireless network (S548). For example, if abnormality such as blue screen occurs during recording of the ultrasonic image, the ultrasonic image recording apparatus 200 transmits the above result to the server 400. As a result, a manager (not shown) of the server 400 may determine whether to maintain or repair the ultrasonic image recording apparatus 200 on the basis of the checked result received from the ultrasonic image recording device 200.

The above description has been given with regard to a case where the quality of the ultrasonic image is higher than the reference quality and recording is executed when motion is detected in the ultrasonic image. Some of the steps shown in FIG. 6 may be omitted. For example, if only items related to the quality of the ultrasonic image are set on the basis of the ultrasonic image recording process, step S543 may be omitted. In another example, only items on whether or not motion is detected in the ultrasonic image are set on the basis of the ultrasonic image recording process, step S542 may be omitted.

As such, a method for recording an ultrasonic image according to an embodiment of the present invention has been described with reference to FIGS. 5 and 6. Some of the steps shown in FIGS. 5 and 6 may be omitted. For example, step S530 of outputting the guide message announcing that the ultrasonic image is automatically recorded in FIG. 5 may be omitted. Likewise, step S545 of outputting the guide message announcing that the recording of ultrasonic image is not executed in FIG. 6 may also be omitted.

As such, the embodiments of the present invention have been described above. In addition to the above-described embodiments, other embodiments of the present invention may also be realized through a medium including a computer readable code/instruction for controlling at least one processing element stated in the above-described embodiments, for example, a computer-readable medium. The medium may correspond to a medium/media enabling storage and/or transmission of the computer readable code.

The computer readable code may be recorded on a medium and also be transmitted through the Internet. More particularly, the medium may include, for example, a recording medium such as a magnetic storage medium (e.g., a read-only memory (ROM), a floppy disk, a hard disk, etc.) and an optical recording medium (e.g., CD-ROM, Blu-Ray, DVD, etc.), a transmission medium such as a carrier wave, or the like. The medium may be a distributed network, and therefore, the computer readable code may be stored/transmitted and executed in a distributed manner. Furthermore, as an example, the processing element may comprise a processor or a computer processor, and the processing element may be distributed and/or included in a single device.

While preferred embodiments of the present invention have been described with reference to the drawings, it will be apparent to one skilled in the art that other concrete modifications may be provided without departing from the spirit and scope of the present invention. Therefore, it would be understood that the embodiments described above are provided for illustration purpose only in every aspect, without particularly limiting the present invention.

1. An ultrasonic image recording apparatus, comprising:
a recognition unit that recognizes identification information of a patient;
a first communication unit that is electrically coupled to an ultrasonic image diagnostic device and receives an ultrasonic image from the ultrasonic image diagnostic device;
a control unit that, when a recording execution comment for recording the ultrasonic image is not input within a reference time, automatically records the ultrasonic image; and
a second communication unit that transmits the recorded ultrasonic image and the identification information mapped to the ultrasonic image, to a server.

2. The apparatus according to claim 1, wherein the control unit executes the recording of the ultrasonic image when a quality of the ultrasonic image is greater than or equal to a reference quality, and then, outputs a guide message informing that the ultrasonic image is automatically recorded.

3. The apparatus according to claim 2, wherein the control unit determines that the quality of the ultrasonic image is greater than or equal to the reference quality when speckle noise is not contained in the ultrasonic image or a boundary between tissues is clearly displayed in the ultrasonic image.

4. The apparatus according to claim 1, wherein the control unit executes the recording of the ultrasonic image when motion is detected in the ultrasonic image, and then, outputs a guide message informing that the ultrasonic image is automatically recorded.

5. The apparatus according to claim 1, wherein the recognition unit includes a bar code reader for recognizing the identification information from a bar code possessed by the patient; and at least one among a first device possessed by the patient and a wireless signal transceiver for transmitting and receiving a wireless signal so as to obtain the identification information.
